(19) United States
(12) Patent Application Publication
(10) Pub. No.: US 2012/0040857 A1
(43) Pub. Date: Feb. 16, 2012

(54) ISOLATION OF FACTORS THAT ASSOCIATE DIRECTLY OR INDIRECTLY WITH CHROMATIN

(75) Inventors: Robert Kingston, Arlington, MA (US); Jerome Dejardin, Prades Le Lez (FR); Matthew Simon, East Boston, MA (US)

(73) Assignee: THE GENERAL HOSPITAL CORPORATION, Boston, MA (US)

(21) Appl. No.: 13/201,476

(22) PCT Filed: Feb. 12, 2010
(86) PCT No.: PCT/US10/24009

§ 371 Date: Nov. 1, 2011

Related U.S. Application Data
(60) Provisional application No. 61/152,357, filed on Feb. 13, 2009.
(60) Provisional application No. 61/225,261, filed on Jul. 14, 2009.

(51) Int. Cl. C40B 30/04; C07H 21/04; C07H 1/06; C07K 2/00; C12Q 1/68; C07H 21/02

(52) U.S. Cl. 506/9; 435/6.13; 536/24.31; 536/25.4; 530/300

(57) ABSTRACT

Methods for isolating non-coding nucleic acids that are associated with chromatin at a target genomic locus are provided. The methods comprise the steps of obtaining a sample that comprises a target genomic DNA sequence and one or more non-coding nucleic acids associated with that DNA sequence; contacting the sample with at least one oligonucleotide probe that comprises a sequence that is complimentary to and capable of hybridising with at least a portion of the target DNA sequence, wherein the oligonucleotide probe comprises at least one modified nucleotide analogue and wherein the oligonucleotide probe further comprises at least one affinity label; allowing the at least one oligonucleotide probe and the target DNA sequence to hybridise with each other so as to form a probe-target hybrid; isolating the probe-target hybrid from the sample by immobilizing the probe-target hybrid through a molecule that binds to the at least one affinity label; and eluting the one or more non-coding nucleic acids that are associated with the target genomic DNA sequence. Also provided are probes suitable for use in the methods of the invention. The methods and probes of the invention are suited to identification of non-coding RNAs including microRNAs and snoRNAs that are associated with chromatin remodelling.

### CROSS REFERENCE TO RELATED APPLICATIONS

This application claims the benefit of priority under 35 U.S.C. 119(e) of U.S. Provisional Application No. 61/152,357 filed Feb. 13, 2009 and U.S. Provisional Application No. 61/225,261 filed Jul. 14, 2009, the contents of which are incorporated herein by reference in their entirety.

### FIELD

The invention relates to assays for nucleic acid factors that associate with defined regions of genomic DNA in the context of chromatin. The invention also relates to probes for use in assays for nucleic acid and polypeptide factors that associate with defined regions of genomic DNA

### BACKGROUND

Epigenetics concerns the transmission of information from a cell or multicellular organism to its descendants without that information being encoded in the nucleotide sequence of genes. Epigenetic mechanisms can operate through chemical modification of the DNA or through post translational modifications of factors that associate with the DNA.

In the genomes of eukaryotic cells, DNA is associated with protein complexes that assist in regulating gene expression, packaging of the DNA and controlling replication. The myriad of proteins and non-coding nucleic acids, such as RNAs, that are associated with the genome contribute to what is termed chromatin: the nuclear material present in the nucleus of most eukaryotic cells. At various times in the cell cycle the level of packaging (or condensation) of the genomic DNA can vary between a lower packaged state such as before the replication of DNA (G1 Phase) to a more condensed state such as during cell division (M phase) where the genome is packaged into chromosomes. Highly expressed genes also tend to exist in a state of low packaging (so called euchromatic state), whereas silenced genes exist in a state of high packaging (so called heterochromatic state). The relative state of condensation, maintenance of this state and the transition between heterochromatin and euchromatin is believed to be mediated largely by a plurality of specialist proteins, polypeptide complexes, and RNAs.

At a fundamental level, the most ‘open’ or euchromatic form of chromatin comprises short sections of the genomic DNA wound around an octet of histone proteins that together form a nucleosome. The nucleosomes are arrayed in series to form a beads-on-a-string structure. Interactions between adjacent nucleosomes allow the formation of more highly ordered chromatin structures. It is these interactions that can be mediated by enzymes that catalyse post-translational modifications of histones, or structural proteins and short non-coding oligonucleotides and polynucleotides that physically interact with and assist in anchoring the histones together.

Epigenetic controls over chromatin organisation and stability are essential for the normal and healthy functioning of a cell. Aberrant epigenetic modifications and a decrease in chromatin stability are often seen in senescent, apoptotic or diseased cells, particularly in cancer cells. It is of considerable importance to identify and characterise the multiple factors that are capable of exhibiting epigenetic activities, as well as those that are capable of interacting with chromatin and chromatin associated proteins. It would also be of great value to identify and characterise novel chromatin associated factors, not least to facilitate a better understanding of chromatin biology as a whole.

Conventionally, isolation of factors associated with genomic DNA has been achieved by performing a chromatin immunoprecipitation (ChIP). In a typical ChIP assay nucleic acid binding proteins and factors are crosslinked to DNA with formaldehyde in vivo. The chromatin is then sheared into small fragments and purified. The purified chromatin fragments are probed with antibodies specific to a known target chromatin binding protein so as to isolate the complex by immunoprecipitation. The precipitated chromatin is treated to reverse the cross-linking, thereby releasing the genomic DNA for sequence analysis. Although it is possible to investigate the ancillary associated factors pulled down by the cross-linking, the method is not restricted to one genomic region and is not optimised for this. Protocols for performing ChIP are disclosed in Nelson et al. (Nature Protocols (2006) 1:179-185) and Crane-Robinson et al. (Meth. Enzym. (1999) 304:533-547). A significant drawback with ChIP based techniques is that for a given sequence, at least one specific protein associated with that sequence must be known already.

There is increasing evidence that dynamic remodelling of chromatin, is also regulated by RNA signalling. Although the precise molecular mechanisms are not yet well understood, they appear to involve the differential recruitment of generic chromatin modifying complexes and DNA methyltransferases to specific loci by specialist non-coding RNAs. Recent studies have shown that RNA polymerase II (RNAPII) transcription of non-coding RNAs is required for chromatin remodelling at the fission yeast Schizosaccharomyces pombe fbp1(+) locus during transcriptional activation (Hirota et al. Nature (2008) November 6; 456 (7218):130-4). The chromatin at fbp1(+) is progressively converted to an open configuration, as several species of non-coding RNAs are transcribed through fbp1(+). A non-coding RNA has also been shown to modulate histone modification and mRNA induction in the yeast GAL gene cluster. GAL10-non-coding RNA transcription recruits the methyltransferase Set2 as well as histone deacetylation activities to this locus in cis, leading to stable changes in chromatin structure (Houseley et al. Mol. Cell. (2008) December 5; 32 (5):685-95). In studies into the roles of long non-coding RNA sequences in mouse pluripotent stem cells two novel developmentally regulated non-coding RNAs, Evx1as and Hoxb5/6as, were observed to share similar expression patterns and localization in mouse embryos with their associated protein-coding genes (Dinger et al. Genome Res. (2008) September; 18 (9):1433-45). The authors used chromatin immunoprecipitation (ChIP) in order to show that both non-coding RNAs were associated with trimethylated H3K4 histones and histone methyltransferase MLL1, suggesting their role in epigenetic regulation of these loci during pluripotent stem cell differentiation.

Further evidence of the importance of non-coding RNA in control of chromatin re-modelling and recruitment of epigenetic modifiers to specific loci in the genome is expected. It would be desirable, therefore, to be able to isolate and characterise non-coding nucleic acids, such as RNA, that are associated with particular regions of genomic DNA. In particular, there is a need for a method of isolating polypeptide and/or nucleic acid factors that associate directly or indirectly with a specified target nucleic acid sequence. In effect, there is a need for a method of chromatin associated isolation that is nucleic acid sequence driven rather than antigen driven which is the case in ChIP. Also, in ChIP a lack of immunoprecipitation does not necessarily reflect an absence of the tested factor, so there is always a risk of false negative results with this technique.

An alternative method to ChIP for isolating polypeptides factors that can associate with specific sequences in genomic DNA (or other cellular nucleic acids) is described in the present inventors' co-pending International Patent application no. PCT/GB2008/002821 (published as WO-A-2009/024781). This technique is referred to as proteomics of intact chromatin (PICh) and provides for a nucleic acid hybridisation driven methodology for identifying proteins that associate directly or indirectly with a given target nucleic acid sequence. The process involves the use of oligonucleotide probes that contain at least one locked nucleic acid nucleotide as well as an affinity label.

It would be desirable to expand the utility of PICh to other chromatin associated factors such as RNAs. It would also be desirable to increase the number and nature of the probes used in PICh-type analysis in order to introduce alternative strategies for targeting regions of chromatin that have especially low levels of sequence repetition. Indeed, identifying polypeptides and ncRNAs that associate with single copy sequence targets (such as single copy genes) within the genome can represent a considerable challenge for both PICh and even more so for ChIP. Hence, there is a need for improved reagents than can effectively penetrate heterochromatic regions and/or hybridise with low copy number sequences within the genome.

### SUMMARY

The present invention overcomes the deficiencies in the art by providing a novel method for isolating nucleic acid factors that associate directly or indirectly with a given target nucleic acid sequence, typically a region of chromatin. Hence, the method of the present invention is hereafter referred to as RNAomics of intact chromatin (RICh). In particular the method of the invention overcomes the aforementioned problems with regard to isolating novel non-coding RNAs that are associated with recruitment of epigenetic modifying factors and remodelling of chromatin structure. The present invention overcomes the deficiencies in the art by providing novel probes, reagents and methods that expand the use of PICh-type analysis of complex genomic targets.

In a first aspect the invention provides a method for isolating one or more non-coding nucleic acids associated with a target DNA sequence that is comprised within chromatin, comprising the steps of:
(a) obtaining a sample that comprises a target DNA sequence as well as one or more non-coding nucleic acids that are associated with the target DNA sequence;
(b) contacting the sample with at least one oligonucleotide probe that comprises a sequence that is complimentary to and capable of hybridising with at least a portion of the target DNA sequence, wherein the oligonucleotide probe comprises at least one modified nucleotide analogue and wherein the oligonucleotide probe further comprises at least one affinity label;
(c) allowing the at least one oligonucleotide probe and the target DNA sequence to hybridise with each other so as to form a probe-target hybrid;
(d) isolating the probe-target hybrid from the sample by immobilizing the probe-target hybrid through a molecule that binds to the at least one affinity label; and
(e) eluting the one or more non-coding nucleic acids that are associated with the target DNA sequence.

A second aspect of the invention provides for a method of screening for a modulator of epigenetic activity comprising:
isolating a non-coding nucleic acid that is identified as associating with a specific region of chromatin in the genome of a eukaryotic cell according to the methods of described herein;
contacting the isolated non-coding nucleic acid with one or more compounds from a library of compounds; and
identifying those compound(s) that bind to and modulate the activity of the isolated non-coding nucleic acid as modulators of epigenetic activity.

A third aspect of the invention provides a method of characterising the biological activity of a non-coding nucleic acid comprising the steps of:
isolating a non-coding nucleic acid that is identified as associating with a specific region of chromatin in the genome of a eukaryotic cell according to the methods described herein;
generating an antisense nucleic acid sequence that is complementary to all or a part of the sequence for the a non-coding nucleic acid;
introducing the antisense nucleic acid sequence into a eukaryotic cell so as to deplete the endogenous level of the non-coding nucleic acid in the cell; and
analysing the phenotype of the eukaryotic cell so as to determine the biological activity of the a non-coding nucleic acid.

A further aspect of the invention provides a nucleic acid analogue probe, suitable for use in PICh/RICh, as set out in formula I below:

A—[C]ₙ—X I

wherein A includes one or more affinity labels tethered to a nucleotide analogue X by a spacer group C of n atoms in length; A comprises a hapten or an immuno-tag; and wherein the nucleotide analogue X is selected from a peptide nucleic acid (PNA); a 2′ modified ribonucleotide analogue, including 2′-O—R sugar modifications, wherein R is selected from the group consisting of: methyl; ethyl; C₁ to C₅ alkyl; and aryl; and
a 2′ substituted ribonucleotide analogue, including 2′-C and 2′-deoxy-2′-halogeno, suitably 2′-deoxy-2′-fluoro ribonucleotides; and a morpholino nucleotide.

Another aspect of the invention provides a nucleic acid analogue oligonucleotide probe, suitable for use in PICh/RICh, conforming to general formula II, set out below:

B—[C]ₙ—Y II

wherein B is an affinity label that is tethered to oligonucleotide sequence Y via a spacer group C comprising a linear chain of n atoms; the oligonucleotide sequence Y comprising at least 10 nucleotides of which no less than 10% are nucleotide analogues; typically at least 25% of the nucleotides are nucleotide analogues; and optionally up to 100% of the nucleotides are nucleotide analogues. Suitably the nucleotide analogues are selected from 2′ modified ribonucleotide analogues, including 2′-O—R sugar modifications, wherein R is selected from the group consisting of: methyl; ethyl; C₁ to C₅ alkyl; and aryl. Optionally the nucleotide analogues are selected from 2′ substituted ribonucleotide analogues including 2′-deoxy-2′-halogeno, suitably 2′-deoxy-2′-fluoro ribonucleotides.

A further aspect of the invention provides a nucleic acid analogue probe, suitable for use in PICh/RICh, conforming to general formula III, set out below:

B—[C]ₙ—P III

wherein B is an immuno-tag or a hapten that is tethered to an peptide nucleic acid (PNA) sequence P via a spacer group C comprising a linear chain of n atoms. Suitably, the spacer group is linked to the N terminal residue of the PNA, P. Alternatively, the spacer group is linked to the C terminal residue of the PNA, P.

Another aspect of the invention provides a nucleic acid analogue probe, suitable for use in PICh/RICh, conforming to general formula IV, set out below:

B—[C]ₙ-M IV

wherein B is an immuno-tag or a hapten that is tethered to a morpholino oligonucleotide sequence M via a spacer group C comprising a linear chain of n atoms.

Optionally the spacer group is linked to the 5′ nucleotide of the morpholino oligonucleotide sequence M.

A yet further aspect of the invention provides a nucleic acid analogue probe, suitable for use in PICh/RICh, conforming to general formula V, set out below:

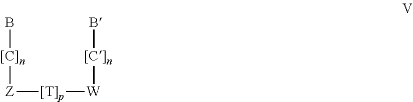

wherein B and B′ comprise an affinity label that is the same or different tethered to respective nucleotides Z and W by spacer groups C and C′ of n atoms in length; and

wherein the nucleotides Z and W are separated by an oligonucleotide chain T of p nucleotides in length, where p is between 0 and 40, the nucleotides Z, W and T being selected suitably from a ribonucleotide, a deoxyribonucleotide, a dideoxyribonucleotide and a modified nucleotide analogue,

the modified nucleotide analogue being selected from:
a locked nucleic acid nucleotide (LNA),
a 2′ modified ribonucleotide analogue, including 2′-O—R sugar modifications, wherein R is selected from the group consisting of: methyl; ethyl; C₁ to C₅ alkyl; and aryl; and
a 2′ substituted ribonucleotide analogue, including 2′-C, and 2′-deoxy-2′-halogeno, suitably 2′-deoxy-2′-fluoro ribonucleotides.

In a specific embodiment of the invention nucleotide Z represents the 5′ nucleotide in the oligonucleotide probe.

A further aspect of the invention provides a kit for performing a PICh/RICh type assay, the kit comprising a probe as described herein, and instructions for use of the probe in a PICh/RICh type assay.

### DRAWINGS

FIG. 1 shows a graphical representation of the PICh/RICh method for identifying factors that can associate with a given genomic sequence.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs. It will be understood that the present invention involves use of a range of conventional molecular biology techniques, which can be found in standard texts such as Sambrook et al. (Sambrook et al (2001) Molecular Cloning: A Laboratory Manual; CSHL Press, USA).

In setting forth the detailed description of the invention, a number of definitions are provided that will assist in the understanding of the invention.

The term “non-coding nucleic acid” as used herein, refers to an oligonucleotide or polynucleotide sequence that is not destined to undergo translation so as to give rise to a corresponding polypeptide sequences. The non-coding nucleic acid sequence may comprise ribonucleic acid (RNA) or deoxyribonucleic acid (DNA), more typically the non-coding nucleic acid sequences of the invention comprise non-coding RNA (ncRNA). As used herein, the term “non coding RNA” (ncRNA) denotes ribonucleic acid polynucleotide transcripts that do not encode a polypeptide product according to the standard dogma of DNA>RNA>protein. Typically, ncRNAs are not messenger RNAs (mRNAs) and may contribute to a variety of epigenetic regulatory effects (Mattick J S (2009) PLoS Genet. 5 (4)). The ncRNAs Air and Xist are well characterised as having cis-acting repressive effects on gene expression in their respective loci.

The methods and compositions of the invention are suitable for isolation of non-coding nucleic acids including: small ncRNAs, antisense polynucleotide sequences (including antisense RNAs), small interfering RNAs (siRNAs), small nucleolar RNAs (snoRNAs), guide RNAs (gRNAs), micro RNAs (miRNAs), Piwi-interacting RNAs (piRNAs) and the polynucleotide products of RNA genes (such as Xist). Stated in another way, the methods and compositions of the present invention are capable of isolating any non-coding nucleic acid that will bind to or interact with chromatin or chromatin associated polypeptides at one or more given loci in the genome.

The term “polypeptide” as used herein, refers to a polymer of amino acid residues joined by peptide bonds, whether produced naturally or in vitro by synthetic means. Polypeptides of less than approximately 12 amino acid residues in length are typically referred to as a “peptide”. The term “polypeptide” as used herein denotes the product of a naturally occurring polypeptide, precursor form or proprotein. Polypeptides also undergo maturation or post-translational modification processes that may include, but are not limited to: glycosylation, proteolytic cleavage, lipidization, signal peptide cleavage, propeptide cleavage, phosphorylation, ubiquitylation, sumoylation, acetylation, methylation and such like. A “protein” is a macromolecule comprising one or more polypeptide chains.

A “polypeptide complex” as used herein, is intended to describe proteins and polypeptides that assemble together to form a unitary association of factors. The members of a polypeptide complex may interact with each other via non-covalent or covalent bonds. Typically members of a polypeptide complex will cooperate to enable binding either to DNA or to polypeptides and proteins already associated with or bound to DNA (i.e. chromatin). Chromatin associated polypeptide complexes may comprise a plurality of proteins and/or polypeptides which each serve to interact with other polypeptides that may be permanently associated with the complex or which may associate transiently, dependent upon cellular conditions and position within the cell cycle. Hence, particular polypeptide complexes may vary in their constituent members at different stages of development, in response to varying physiological conditions or as a factor of the cell cycle. By way of example, in animals, polypeptide complexes with known chromatin remodelling activities include Polycomb group gene silencing complexes as well as Trithorax group gene activating complexes.

Polypeptide complexes may also comprise one or more non-coding nucleic acids, for example one or more non-coding RNAs. There is increasing evidence that recruitment and targeting of chromatin binding proteins can be mediated via non-coding nucleic acids. It has been shown, for example, that a ncRNA cofactor recruits Polycomb complexes to their target locus (Zhao et al., Science. (2008) Oct. 31; 322 (5902):750-6).

The term “isolated”, when applied to a nucleic acid or polypeptide sequence is a sequence that has been removed from its natural organism of origin. Typically, an isolated polypeptide or polynucleotide/nucleic acid molecule has been removed from the environment in which it was produced; although, it is not necessarily in a pure form. That is, an isolated polypeptide or polynucleotide is not necessarily 100% pure, but may be about 10%, 20%, 30%, 40%, 50%, 60%, 70%, 80% or 90% pure. A purified, isolated polypeptide or polynucleotide is advantageously at least 80% pure, and may be at least 90%, at least 95% or at least 98% pure (e.g. 99% pure). In the present context, the term “isolated” when applied to a polypeptide is intended to include the same polypeptide in alternative physical forms whether it is in the native form, denatured form, dimeric/multimeric, glycosylated, crystallised, or in derivatised forms. Advantageously, the nucleic acid molecules/polynucleotides/oligonucleotides (e.g. nucleic acid probes, ncRNA, siRNA molecules etc.), and polypeptides/peptides (e.g. antibodies or fragments thereof) of the invention are isolated; and more beneficially, purified.

Chromatin is the compacted structure of genomic DNA present in the nucleus of most eukaryotic cells. It comprises DNA and a plurality of DNA-binding proteins as well as certain non-coding nucleic acids such as ncRNAs. The term ‘chromatin’ derives from the readiness of this cellular material to hold stain with certain chemical dyes (chromaticity). Chromatin is primarily comprised of DNA associated with histone proteins that together form a basic nucleosomal structure. The nucleosome comprises an octet of histone proteins around which is wound a stretch of double stranded DNA 146 bp in length. Histones H2A, H2B, H3 and H4 are part of the nucleosome while histone H1 can act to link adjacent nucleosomes together into a higher order structure. Assembly into higher order structures allows for greater packing, or condensation of the DNA. Chromatin is often referred to as occurring in two main states, euchromatin and heterochromatin, corresponding to uncondensed actively transcribed DNA and condensed DNA respectively. Many further polypeptides and protein complexes interact with the nucleosome and the histones in order to mediate transition between the euchromatic and heterochromatic states. The identity and functional activity of many of these crucially important chromatin associated proteins and complexes is presently unknown.

Epigenetics concerns the transmission of information from a cell or multicellular organism to its descendants without that information being encoded in the nucleotide sequence of genes. Epigenetic controls are typically established via chemical modification of the DNA or chromatin structure. Gene expression can be moderated, in some cases, via the covalent attachment of chemical groups to polypeptides that are associated with or that can bind to DNA. By way of example, methylation, sumoylation, phosphorylation, ubiquitylation and/or acetylation of histones can lead to activation or silencing of gene expression in the region of the genome where these epigenetic modifications have occurred. Epigenetic modifications can occur at different times in the normal development of an organism, and also during transformation of normal cells into cancerous cells. Such modifications often result in the silencing or activation of certain genes. In cancer, it is well documented that the majority of tumour cells display abnormal DNA epigenetic imprints (Feinberg A P & Vogelstein B, (1983) Nature 1 (5895):89-92).

The term “cancer” is used herein to denote a tissue or a cell located within a neoplasm or with properties associated with a neoplasm. Neoplasms typically possess characteristics that differentiate them from normal tissue and normal cells. Among such characteristics are included, but not limited to: a degree of anaplasia, changes in cell morphology, irregularity of shape, reduced cell adhesiveness, the ability to metastasise, increased levels of angiogenesis, increased cell invasiveness, reduced levels of cellular apoptosis and generally increased cell malignancy. Terms pertaining to and often synonymous with “cancer” include sarcoma, carcinoma, tumour, epithelioma, leukaemia, lymphoma, polyp, transformation, neoplasm and the like.

By the term “modulator” it is meant a molecule (e.g. a chemical substance/entity) that effects a change in the activity of a target molecule (e.g. a gene, enzyme etc.). The change in activity is relative to the normal or baseline level of activity in the absence of the modulator, but otherwise under similar conditions, and it may represent an increase or a decrease in the normal/baseline activity. The modulator may be any molecule as described herein, for example a small molecule drug, an antibody or a nucleic acid. In the context of the present invention, the target is a novel chromatin associated factor, optionally comprising a non-coding nucleic acid, that has been identified according to screening method of the invention. The modulation of chromatin-associated factors may be assessed by any means known to the person skilled in the art; for example, by identifying a change in the expression of genes regulated by the chromatin associated factor.

An embodiment of the present invention resides in the development of a method for identifying non-coding nucleic acids that are associated with a particular target chromatin site, gene or stretch of genomic nucleic acid, such as DNA. The method utilises a high specificity nucleic acid probe labelled with an affinity tag that allows for isolation of probe-target hybridised sequences. The method of the invention demonstrates considerable advantage over immuno-precipitation based techniques, such as ChIP, which rely on the presence of a known protein antibody target that is already bound to the DNA in order to pull down any associated unknown non-coding nucleic acid sequences. The method of the present invention demonstrates an advantage of enabling the identification of any and all DNA and/or chromatin associated non-coding nucleic acid sequences (such as ncRNAs) at a specified target site without the need for prior knowledge of any of the proteins that may or may not be present at that site. Hence, also, if the antibody is quantitatively precipitating a crosslinked antigen, which is rare, ChIP does not permit purification of a single loci but a mixture of loci that contain the protein of interest. The method of the invention also allows for changes in chromatin/DNA associated non-coding nucleic acid sequences to be monitored under different cellular conditions as well.

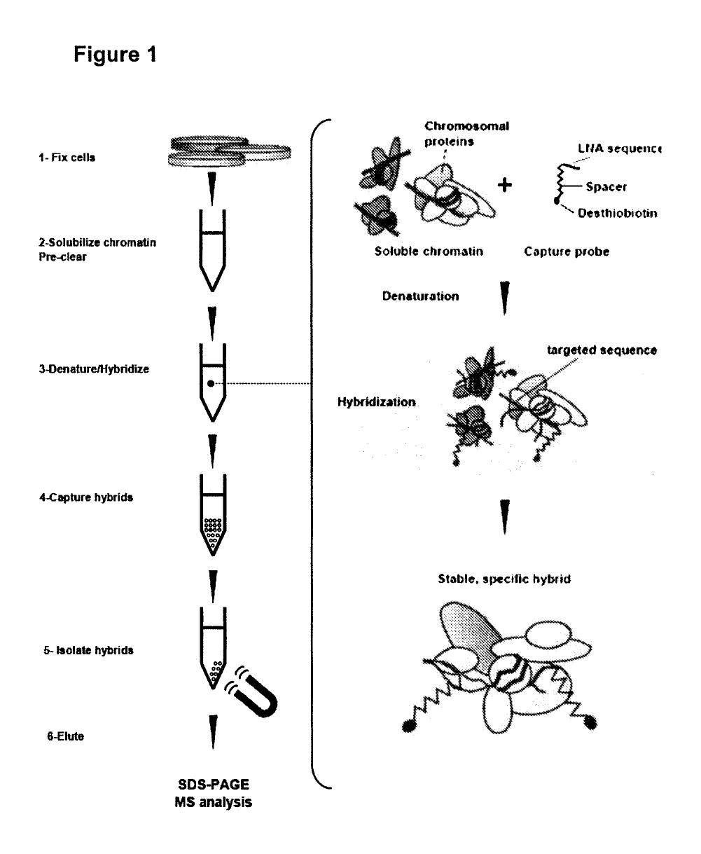

A specific embodiment of the invention is outlined in FIG. 1. In brief, cells are fixed, the chromatin solubilized, a specific probe is hybridized to the chromatin, the hybridized chromatin is then captured on magnetic beads, the hybrids are eluted and the polypeptide factors or non-coding RNAs identified. Extensive crosslinking with agents such as formaldehyde can be used to preserve protein-DNA, protein-protein and protein-non-coding nucleic acid interactions. Unlike strategies based upon antibody antigen affinity, nucleic acid hybridization is insensitive to the presence of ionic detergents, which allows the use of these detergents throughout to limit contamination. In this particular embodiment of the invention, to increase the stability of the probe-chromatin interactions, Locked Nucleic Acid (LNA) containing oligonucleotides have used as probes because LNA residues have an altered backbone that favours base stacking thereby significantly increasing their melting temperature (Vester, B., and Wengel, J. (2004) Biochemistry 43, 13233-13241). As discussed in more detail below, LNAs are simply one of a number of suitable ways to achieve improved hybridisation performance. To minimize the steric hindrance (which is detrimental for yields) observed upon immobilization of chromatin a very long spacer group is placed between the immobilization tag and the LNA probe. Suitable spacers include long chain aliphatic groups, or spacers can be synthesised from methoxyoxalamido and succinimido precursors such as those described in Morocho, A. M. et al (Methods Mol Biol (2005) 288, 225-240), or suitably using a polyethylene glycol (PEG) based linker Finally the co-elution of non-specific factors is limited by using desthiobiotin, a biotin analogue with weaker affinity for avidin, permitting a competitive gentle elution using biotin.

An embodiment of the present invention resides in the development of improved probes for identifying non-coding nucleic acids and/or polypeptide factors that are associated with a particular target chromatin site, gene or stretch of genomic nucleic acid, such as DNA. The method utilises a high specificity nucleic acid probe labelled with an affinity tag that allows for isolation of probe-target hybridised sequences. The method, known as PICh (when applied to isolation of proteins) or RICh (when applied to isolation of ncRNAs) demonstrates considerable advantage over immuno-precipitation based techniques, such as ChIP, which rely on the presence of a known protein antibody target that is already bound to the DNA in order to pull down any associated unknown non-coding nucleic acid sequences. PICh and RICh demonstrate an advantage of enabling the identification of any and all polypeptides, DNA and/or chromatin associated ncRNAs at a specified target site without the need for prior knowledge of any of the proteins that may or may not be present at that site. Hence, also, if the antibody is quantitatively precipitating a crosslinked antigen, which is rare, ChIP does not permit purification of a single loci but a mixture of loci that contain the protein of interest. PICh and RICh also allow for changes in chromatin/DNA associated non-coding nucleic acid sequences to be monitored under different cellular conditions as well.

In a further specific embodiment of the invention, non-coding nucleic acid molecules associated with telomere sequences in telomerase positive cancer cells and in an ALT (alternative lengthening of telomeres) cancer cell type were identified according to the method of the invention. In addition to the proteins and protein complexes expected to be present and associated to the telomere sequences, a surprising and unexpected number of additional polypeptides and a non-coding nucleic acid were identified using the present techniques as with the associated proteomic technique of PICh (described in the inventors' co-pending international patent application no. PCT/GB2008/002821). Some of the chromatin associating factors included known proteins and non-coding nucleic acids not previously expected to associate with chromatin, whereas other known chromatin associated factors were newly identified as localising to telomeres. Hence, the present invention further provides for identification and isolation of non-coding nucleic acids, such as ncRNAs, with a novel chromatin association activity.

Accordingly, the method of the present invention can comprise a first step, in which an affinity labelled nucleic acid analogue probe sequence specifically hybridises to a preparation of sample chromosomal material comprising the chromatin target of interest. The probe is designed such that it hybridises with a specified target sequence in the genomic DNA. The target sequence may suitably include a unique gene sequence, a repetitive sequence, or a sequence known to have a role in higher order structural formation. The probe sequence is introduced at a concentration and under stringent hybridisation conditions such that only the target sequence in the genomic DNA is bound.

A specific embodiment of the present invention further resides in the provision of a subset of non-coding nucleic acids, including ncRNAs, that are newly identified as possessing chromatin association activity and which potentially act as novel epigenetic factors.

In a specific embodiment, the present invention provides a method by which protein complexes associated with chromatin at a specified site in the genome and which may include non-coding nucleic acids can be characterised. It should be noted that the method of the invention is not limited to those complexes that are solely DNA binding, but includes associated complexes such as those with histone binding activity, for example.

In more detail, the method of the invention can comprise a first step, in which an affinity labelled nucleic acid probe sequence specifically hybridises to a preparation of sample chromosomal material comprising the chromatin target of interest. The probe is designed such that it hybridises with a specified target sequence in the genomic DNA. The target sequence may suitably include a unique gene sequence, a repetitive sequence, or a sequence known to have a role in higher order structural formation. The probe sequence is introduced at a concentration and under stringent hybridisation conditions such that only the target sequence in the genomic DNA is bound.

The probe is labelled with one or more suitable affinity tags. Affinity tags may include immuno-tags or haptens. For example, one or more of the nucleotides contained within the probe sequence may be biotinylated (either with biotin or a suitable analogue thereof—e.g. desthiobiotin). Alternative affinity labels may include digoxigenin, dinitrophenol or fluorescein, as well as antigenic peptide ‘tags’ such as polyhistidine, FLAG, HA and Myc tags. For target sequences that are present in high copy number in the sample of interest, probes will typically comprise only a single type of affinity label. For targets of low concentration, such as single copy sequences in the genome of an organism, optionally the oligonucleotide probes of the invention may comprise more than one type of tag. By way of example, in an embodiment of the invention an oligonucleotide probe directed towards a single copy promoter region in a eukaryotic cell comprises modified nucleotides labelled with both desthiobiotin and digoxigenin. The inclusion of more than one affinity tag in the probes of the invention can significantly increase the sensitivity of the process for low copy number targets.

The probe nucleic acid sequences previously known for use in PICh and RICh comprise one or more locked nucleic acid (LNA) nucleotides. LNA nucleotides are bicyclic RNA analogues that contain a 2′-O, 4′-C methylene bridge in the ribose moiety. The methylene bridge restricts the flexibility of the ribofuranose ring and ‘locks’ the structure into a rigid C3-endo conformation. By altering the conformation of the nucleotide in this way, the resultant oligomer/polymer probe demonstrates enhanced hybridization performance and biostability. LNA containing probe sequences have demonstrated suitability for ISH applications (Silahtoroglu et al. (2004) Cytogenet Genome Res 107:32-37) and techniques for probe sequence design are described in Tolstrup et al. (Nucleic Acids Res. 2003 Jul. 1; 31 (13): 3758-3762).

The present invention relates in part to the synthesis and use of novel probes with optimal utility in RICh and PICh techniques. The probes comprise nucleic acid analogues that serve to elevate the Tₘ of the probe-target duplex hybrid to a level in excess of that seen for a corresponding probe containing only naturally occurring ribonucleotides (RNA) or deoxyribonucleotides (DNA). More specifically, the invention provides probes that comprise modified, non-naturally occurring or otherwise synthetic, analogues of ribonucleotides and deoxyribonucleotides together with one or more affinity labels, the affinity label being separated from the backbone of the oligo probe via an extra-long spacer group. Typically, in the case of synthetic nucleic acid analogues comprising a ribonucleotide core structure the spacer group is added at the 2′ position on the ribose ring (e.g. see Morocho et al., supra). For analogues that do not conform to a conventional ribonucleotide core structure, the spacer group is attached at a position that will not inhibit or interfere with hybridisation between the probe and the target nucleic acid sequence. For instance, where a conventional peptide nucleic acid (PNA) probe containing N-(2-aminoethyl)glycine units is utilised, the spacer group may be attached via linkage to the N or C terminus of the PNA sequence. Alternative or derivatized PNA based probes that incorporate one or more functionalised substitutions in addition to the core N-(2-aminoethyl)glycine backbone may be adapted to include the extra-long spacer at positions within the PNA probe in addition to or in place of an N or C terminal linkage. Examples of such derivatized PNA sequences are known in the art (for example see, Wojciechowski F. & Hudson R. H. (2007) Curr Top Med. Chem.; 7 (7):667-79; and Pensato S. et al. (2007) Expert Opin Biol Ther. August; 7 (8):1219-32).

Oligonucleotide probes comprising nucleic acid analogues of the present invention may also comprise one or more modified nucleotides that comprise various backbone and 2′-0 sugar modifications including but not limited to 2′-O-methyl (2′-OMe), 2′-O-methoxyethyl (2′-MOE), and 2′ deoxy-2′ fluoro (2′F) ribonucleotides. A range of alternative 2′ modified ribonucleotides, including various alkyl modifications, may also be incorporated into the probes of the invention, for example see Egli et al. (2005) Biochemistry June 28; 44 (25):9045-57.

Protein nucleic acids (PNAs) are non-naturally occurring analogues of nucleic acids in which the phosphodiester backbone of the polymer is replaced with an polyamide made up of N-(2-aminoethyl)glycine units. Preparation and uses of PNA are extensively described in European Patent No. 0586474 (see also Nielsen et al. (1991) Science. December 6; 254 (5037):1497-500; and Egholm, M. et al. (1993) Nature, 365, 566-568). PNAs can hybridise with single stranded nucleic acids at very high affinity and at lower salt concentrations, thereby facilitating high stringency binding under less conducive conditions than would be seen for corresponding RNAs or DNAs. In accordance with the present invention, PNA oligo probes of between 10 and 100 nucleobases in length can be used, suitably between 12 and 70 nucelobases in length, even more suitably between 15 and 50 nucleobases in length. In an example of the invention in use, the PNA oligo is directed towards a telomere specific sequence and corresponds substantially to that shown in SEQ ID NO:1 below:

[SEQ ID NO: 1]
5′ ttagggttagggttagggttagggt 3′

The PNA probes of the invention may comprise an N-terminally located extra-long spacer of around 100 atoms in length to which at its other end is tethered a desthiobiotin group.

A further alternative nucleotide analogue suitable for use in the probes of the invention includes morpholino oligomers. Morpholino nucleotide analogues include standard nucleic acid bases which are bound to a morpholine ring instead of to a ribose ring (Summerton J. & Weller D. (1997) Antisense Nucleic Acid Drug Dev. June; 7 (3):187-95). Typically morpholino nucleotides are linked to each other via phosphorodiamidate groups instead of phosphates. Oligomeric morpholine probes bind to complementary sequences of a target nucleic acid sequence by standard nucleic acid base-pairing. Synthesis of morpholino containing oligomers is described in EP-0506830 and WO-A-86/05518 and morpholino oligonucleotides are available from Gene Tools LLC (Philomath, Oreg., USA). In accordance with the present invention, morpholino probes of between 10 and 100 nucleotides in length can be used, suitably between 12 and 70 nucleotides in length, even more suitably between 15 and 50 nucleotides in length. In an example of the invention in use, the morpholino oligo probe is directed towards a telomere specific sequence and corresponds substantially to that shown in SEQ ID NO:1. The morpholino probe comprises a single 5′ located methoxyoxalamido extra-long spacer of around 100 atoms in length to which at its other end is tethered a desthiobiotin group.

Prior to the hybridisation step, the chromatin can be partially enzymatically digested in order to increase the resolution and to facilitate the next step of the method, which involves ‘pull-down’ of the probe-target sequence hybrid. Alternatively, the chromatin can be fragmented by physical methods such as ultrasonication, or by a combination of physical and enzymatic approaches.

The ‘pull-down’ step is facilitated by use of a binding moiety that engages the affinity tag and enables the hybridised sequences to be isolated. In case of a biotinylated probe sequence, isolation of the hybridised sequences can be effected in vitro by exposing the hybridised sequences to microbeads coated with streptavidin. In this way the hybridised sequences will bind to the beads and can be precipitated out of solution via a straightforward microcentrifugation step. Alternatively the microbeads may comprise a magnetic component allowing for immobilisation of the beads via exposure to a magnetic field (see FIG. 1). Alternative isolation strategies include the immobilisation of the modified nucleotide containing probes on a solid substrate such as a microarray support or a dipstick. In this way the ‘pull down’ is facilitated by localisation to a specific area on a surface, which can then be suitably adapted for high throughput or automated analysis.

The purified, or ‘pulled-down’ hybridised sequences comprise affinity labelled probe hybridised to the target sequence together with any associated chromatin polypeptides, proteins and non-coding nucleic acids that are bound to the target sequence. These associated chromatin polypeptides, proteins and non-coding nucleic acids can be isolated from the pulled-down material by standard protein precipitation steps and, if required, separated via electrophoretic (e.g. SDS-PAGE) or chromatographic techniques (e.g. HPLC). The protein component of the complex can then be removed via enzymatic digestion to leave the non-coding nucleic acids for analysis and characterisation.

The non-coding nucleic acids can be analysed to determine their identity such as via standard sequencing methodologies (e.g. “Solexa™” high throughput sequencing, Illumina Inc., California, USA) or via use of expression profiling micro-array panels (e.g. microRNA expression profiling). Qualitative changes in the composition of known chromatin associating non-coding nucleic acids can be monitored using microarray technologies that are directed to constituent members of the complexes of interest.

It will be appreciated that the method of the invention is not limited to a specific type of genomic DNA and can be directed a virtually any target sequence in the genome in order to identify the associated non-coding nucleic acids profile. In addition, for any given genomic sequence the method can be employed at different times in development, in the cell cycle or following exposure of the cell to external stimuli. As such, RICh can allow for detailed profiling of the change in non-coding nucleic acids associated with a specific target sequence to be monitored. In combination with PICh the total constituent change in polypeptide/non-coding nucleic acid complexes can be determined in order to track chromatin remodelling in a way that has not been possible before. Moreover, the method of the invention allows for the identification of novel DNA and chromatin associated non-coding nucleic acids or non-coding nucleic acids with previously unknown or appreciated activity. In addition to providing information on the identity of non-coding nucleic acids bound to a locus, the present invention can provide information on the relative levels of abundant non-coding nucleic acids bound to a given sequence in distinct cell types.

In further embodiments of the invention, lower abundance targets are considered. For example, in Drosophila melanogaster (fruit fly) the Fab-7 region represents a single copy chromatin fragment that is responsible for the maintenance of the transcriptional status of a homeotic gene named Abdominal-B. This fragment is named a Polycomb Response Element and recruits factors of the Polycomb and trithorax group of genes. Understanding the function of the Fab-7 site in Drosophila is critical to the understanding of the maintenance of epigenetic states in higher eukaryotes, as the basic chromatin regulation is typically conserved in higher organisms such as mouse and humans. The chromatin associated polypeptides, proteins and non-coding nucleic acids comprised within complexes that bind to Fab-7 are believed to be central to improving the efficiency of stem cell therapies and may be subject to aberrant regulation in several cancers. Fab-7 is a more complex sequence to target compared to telomeres which are highly repetitive. As a result, Fab-7 requires the use of several distinct probes to cover the region in an overlapping or contiguous fashion, unlike for telomeres where a single probe can hybridize along the length of the targeted region.

A further embodiment of the invention targets the HIV promoter (located in the LTR of the pro-virus), which is integrated into the genome of an infected human T cell line. Understanding the regulation of this promoter is crucial in determining the aetiology and molecular mechanisms of HIV/AIDS. In particular, it is known that the pro-virus can remain silent for periods of time and it is critical to understand why reservoirs of infected cells escape therapies, because those cells contain so called ‘dormant’ virus. Determining the relevant epigenetic components, including associated non-coding nucleic acids, and the state of the chromatin in the region of the HIV LTR promoter allows further insight into the mechanisms of disease and thereby identifies new candidate drug targets for HIV therapy. The invention allows for comparisons to be made between chromatin composition at the HIV LTR region in human T cell lines that model the two situations dormant or active, and this also provides important data that is useful in designing novel drug treatments for HIV/AIDS.

The nucleic acid analogue probe, in specific embodiments of the invention, comprises at least one group that conforms to general formula I, set out below:

A—[C]ₙ—X I

wherein A includes an affinity label tethered to a nucleotide analogue X by a spacer group C of n atoms in length. Typically A comprises a hapten. In a specific embodiment of the invention A comprises biotin, or an analogue thereof such as a desthiobiotin molecule. The nucleic acid analogue X can be selected suitably from a peptide nucleic acid (PNA); a 2′ substituted nucleic acid analogue; and a morpholino nucleotide. The length of the spacer group is such that n is suitably between about 40 and about 170 atoms; more suitably between about 75 and 130 atoms; more typically between about 90 and about 120 atoms; suitably between about 100 and about 110 atoms.

In a specific embodiment, the nucleic acid analogue oligonucleotide probe of the invention conforms to general formula II, set out below:

B—[C]ₙ—Y II

wherein B is a immuno-tag or a hapten that is tethered to an oligonucleotide sequence Y via a spacer group C comprising a linear chain of n atoms. The oligonucleotide sequence Y comprises at least 10 nucleotides of which no less than 10% are nucleotide analogues; typically at least 25% of the nucleotides are nucleotide analogues; optionally up to 100% of the nucleotides are nucleotide analogues. The nucleotide analogues of the invention are selected from a range of 2′ modified ribonucleotide analogues, including but not limited to 2′-O— modified analogues such as 2′-O—R sugar modifications, wherein R is selected from the group consisting of: methyl; ethyl; C₁ to C₅ alkyl; and aryl. Alternative 2′ substituted ribonucleotide analogues include 2′-deoxy-2′-halogeno, suitably 2′-deoxy-2′-fluoro ribonucleotides; and 2′-C modified analogues. In a specific embodiment of the invention, the oligonucleotide probe is 25 nucleotides in length and comprises around 50% modified nucleic acid analogues and 50% ribonucleotides or deoxyribonucleotides. The length of the spacer group C is such that n is suitably between about 40 and about 170 atoms; more suitably between about 70 and 130 atoms; more typically between about 90 and about 120 atoms; suitably between about 100 and about 110 atoms. Optionally, the spacer group is linked to the 5′ nucleotide of the oligonucleotide Y, with the 5′ nucleotide optionally being a nucleotide analogue.

In a specific embodiment, the oligonucleotide probe of the invention conforms to general formula III, set out below:

B—[C]ₙ—P III

wherein B is a immuno-tag or a hapten that is tethered to an peptide nucleic acid (PNA) sequence P via a spacer group C comprising a linear chain of n atoms. The PNA sequence P comprises at least 10 PNA residues/nucleobases. In a specific embodiment of the invention, the PNA sequence is at least 25 residues in length. The length of the spacer group C is such that n is suitably between about 40 and about 170 atoms; more suitably between about 70 and 130 atoms; more typically between about 90 and about 120 atoms; suitably between about 100 and about 110 atoms. Optionally, the spacer group is linked to the N terminal residue of the PNA, P. Alternatively or additionally, the spacer group is linked to the C terminal residue of the PNA, P.

In a further specific embodiment, the oligonucleotide probe of the invention conforms to general formula IV, set out below:

B—[C]ₙ-M IV

wherein B is a immuno-tag or a hapten that is tethered to a morpholino oligonucleotide sequence M via a spacer group C comprising a linear chain of n atoms. The morpholino oligonucleotide sequence M comprises at least 10 nucleotides. In a specific embodiment of the invention, the morpholino oligonucleotide sequence M is at least 15 nucleotides in length, suitably at least 20 nucleotides in length, more suitably at least 25 nucleotides in length. The length of the spacer group C is such that n is suitably between about 40 and about 170 atoms; more suitably between about 70 and 130 atoms; more typically between about 90 and about 120 atoms; suitably between about 100 and about 110 atoms. Optionally, the spacer group is linked to the 5′ nucleotide of the morpholino oligonucleotide sequence M.

A further specific embodiment of the invention provides an oligonucleotide probe that comprises a group that conforms to general formula V, set out below:

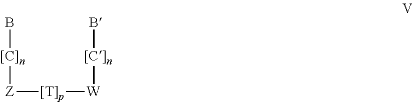

wherein B and B′ include an affinity label that is the same or different tethered to respective nucleotides Z and W by spacer groups C and C′ of n atoms in length; and wherein the nucleotides Z and W are separated by an oligonucleotide chain T of p nucleotides in length, where p is between 0 and 40, the nucleotides Z, W and T being selected suitably from a ribonucleotide, a deoxyribonucleotide, a dideoxyribonucleotide and a modified nucleotide analogue. The modified nucleotide analogue is selected from:
a locked nucleic acid nucleotide (LNA); and/or
a 2′ modified ribonucleotide analogue, including 2′-O—R sugar modifications, wherein R is selected from the group consisting of: methyl; ethyl; C₁ to C₅ alkyl; and aryl; and/or
a 2′ substituted ribonucleotide analogue, including 2′-deoxy-2′-halogeno, suitably 2′-deoxy-2′-fluoro ribonucleotides, or a 2′-C substitution.

In a particular embodiment of the invention the oligonucleotide probe comprises the group of general formula V such that nucleotide Z represents the 5′ nucleotide in the oligonucleotide probe. The probes comprising the group of formula V are particularly suitable for targeting sequences in chromatin that have a low frequency of occurrence, such as single copy number sequences (e.g. particular genes). The separation between nucleotides Z and W is shown by the intervening oligonucleotide chain T, which is suitably anything between 0 and 40 nucleotides in length, preferably between 1 and 30 nucleotides. Typically, nucleotides Z and W at least will be modified nucleotide analogues. Suitably, spacer groups C and C′ may be of the same or different lengths. In embodiments of the invention, the oligonucleotide probe may comprise one or more groups of formula V.

The invention also provides for kits and assay compositions that comprise the above described probes of the invention. Such kits may additionally comprise other components and reagents suitable for use in performing a PICh/RICh type assay such as that shown in FIG. 1, for example an instruction manual, cross linking buffers, hybridisation buffers, antibodies and the like.

The probe nucleic acid sequences may be directed at any sequence that appears in, for example, genomic DNA. Advantageously, the invention is not limited to coding or non-coding sequences, nor is it restricted to use with euchromatic regions of the genome. Where the target sequence comprises a repeat sequence, such as a repeated telomeric sequence, a single probe species is often sufficient to effect pull down. In instances where a unique or less frequently repeated sequence is targeted it may be necessary to use a combination of two or more probes that bind to consecutive, overlapping or closely located regions of the target locus. In an example of the invention in use, purification of mouse pericentric heterochromatin is achieved using a combination of three probe oligonucleotides that hybridise with around to 25% of the target sequence. However, in order to optimise the pull down reaction the more target sequence covered by the probe(s), the greater the yield.

The present invention also relates to methods and compositions for the treatment of diseases associated with modified expression of one or more of the novel chromatin associated factors identified according to the method of the present invention.

Reagents for the inhibition of expression and/or biological activity of a specified chromatin associated factor include, but are not limited to, antisense nucleic acid molecules, siRNA (or shRNA), ribozymes, small molecules, and antibodies or the antigen binding portions thereof. For a review of nucleic acid-based technologies see, for example, Kurreck, J. (2003) “Antisense technologies—Improvement through novel chemical modifications”, Eur. J. Biochem. 270: 1628-1644. The reagents for inhibition of the chromatin associated factor may affect expression and/or biological activity indirectly; for example, by acting on a factor that affects gene expression or that modifies or inhibits the biological activity of the novel chromatin associated factor.

Antisense nucleic acid sequences can be designed that are complementary to and will hybridise with a given non-coding nucleic acid in-vivo. Antisense nucleic acid sequences may be in the form of single stranded DNA or RNA molecules that hybridise to all or a part of the sequence of mRNA for the specified chromatin associated factor. Typically, an antisense molecule is at least 12 nucleotides in length and at least 90%, 93%, 95%, 98%, 99% or 100% complementary to the chosen target nucleotide sequence. Antisense oligonucleotides can be of any reasonable length, such as 12, 15, 18, 20, 30, 40, 50, 100, 200 or more nucleotides, having the advantageous above-mentioned complementarity to its corresponding target nucleotide sequence.

An antisense oligonucleotide may contain modified nucleotides (or nucleotide derivatives), for example, nucleotides that resemble the natural nucleotides, A, C, G, T and U, but which are chemically modified. Chemical modifications can be beneficial, for example, in: providing improved resistance to degradation by endogenous exo- and/or endonucleases, to increase the half-life of an oligonucleotide in vivo; enhancing the delivery of an oligonucleotide to a target cell or membrane; or increasing the bioavailability of an oligonucleotide. Typically, an antisense molecule contains a mixture of modified and natural nucleotides, and in particular, the 5′ most and/or the 3′ most nucleotides (e.g. the two outermost nucleotides at each end of the strand) may be modified to increase the half-life of the antisense molecule in vivo. In addition, or in the alternative, the backbone of an antisense molecule may be chemically modified, e.g. to increase resistance to degradation by nucleases. A typical backbone modification is the change of one or more phosphodiester bonds to a phosphorothioate bonds. An antisense molecule may suitably also comprise a 5′ cap structure and/or a poly-A 3′ tail, which act to increase the half-life of the antisense molecule in the presence of nucleases.

Antisense oligonucleotides can be used to inhibit expression, localisation or activity of one or more chromatin associated factors that comprise an non-coding nucleic acid component identified according to the method of the present invention in target tissues and cells in vivo. Alternatively, such molecules may be used in an ex vivo treatment, or in an in vitro diagnostic test.

Requirements for the design and synthesis of antisense molecules against a specific target non-coding nucleic acid, as well as methods for introducing and expressing antisense molecules in a cell, and suitable means for modifying such antisense molecules are known to the person of skill in the art.

For example, antisense molecules for use in therapy may be administered to a patient directly at the site of a tumour (for example, by injection into the cell mass of the tumour), or they can be transcribed from a vector that is transfected into the tumour cells. Transfection of tumour cells with gene therapy vectors can be achieved, for example, using suitable liposomal delivery systems or viral vectors (Hughes, 2004, Surg. Oncol., 85 (1): 28-35).

Another means of specifically down-regulating a target gene, such as a chromatin associated factor gene is to use RNA interference (RNAi). Naturally, RNAi is typically initiated by long double-stranded RNA molecules, which are processed by the Dicer enzyme into 21 to 23 nucleotides long dsRNAs having two-nucleotide overhangs at the 5′ and 3′ ends. The resultant short dsRNA molecules are known as small interfering RNAs (siRNAs). These short dsRNA molecules are then thought to be incorporated into the RNA-induced silencing complex (RISC), a protein-RNA complex, which acts as a guide for an endogenous nuclease to degrade the target RNA.

It has been shown that short (e.g. 19 to 23 bp) dsRNA molecules (siRNAs) can initiate RNAi, and that such molecules allow for the selective inactivation of gene function in vivo, for example, as described in Elbashir et al. (2001, Nature, 411: 494-498). Thus, this technique provides a means for the effective and specific targeting and degradation of mRNA encoding a chromatin associated factor in cells in vivo. Accordingly, the invention provides siRNA molecules and their use to specifically knock down one or more chromatin associated non-coding nucleic acids identified by the methods of the present invention. Lentiviral vector transfection systems have been proposed for obtaining effective siRNA knock down of microRNAs in the cell (Gentner et al., Nat. Methods. (2009) January; 6 (1):63-6).

As in the case of antisense and ribozyme technology, an siRNA or shRNA molecule for in vivo use advantageously contains one or more chemically modified nucleotides and/or one or more modified backbone linkages.

Pharmaceutical preparations of the invention are formulated to conform to regulatory standards and can be administered orally, intravenously, topically, or via other standard routes. The pharmaceutical preparations may be in the form of tablets, pills, lotions, gels, liquids, powders, suppositories, suspensions, liposomes, microparticles or other suitable formulations known in the art.

Thus, the invention encompasses the use of molecules that can regulate or modulate activity or expression of the novel chromatin associated non-coding nucleic acids of the invention for treating disease. Typically diseases associated with aberrant activity or expression of chromatin-associated non-coding nucleic acids will include: cancer, premature aging, inflammatory disease, autoimmune disease, virally induced diseases and infections and infertility.

Novel chromatin associated factors (non-coding nucleic acids) identified by the methods of the invention can be recombinantly expressed individually or in combination to create transgenic cell lines and purified factors for use in drug screening. Cell lines over-expressing the non-coding nucleic acids can be used, for example, in high-throughput screening methodologies against libraries of compounds (e.g. “small molecules”), antibodies or other biological agents. These screening assays may suitably be either cell-based assays, in which defined phenotypic changes are identified (analogous to calcium signalling in GPCR FLIPR screening), or can serve as the source of high levels of purified proteins for use in affinity-based screens such as radio-ligand binding and fluorescence polarisation.

It is apparent, therefore, that the information derived from the methods of the present invention allows for the accurate identification of a chromatin activity for non-coding nucleic acids in the cell. By providing a cellular context for these diverse factors, as well as information on potential co-factors, the present invention allows for a more focussed approach to drug discovery, target selection and systems biology. The identification of the non-coding nucleic acids, such as ncRNAs, that interact with genomic regions of interest is also critical to the understanding of genome biology. These questions have previously been studied using genetics, chromatin immunoprecipitation, and cell biology. By establishing the ‘chromatin formula’ of factors bound at specific loci and at a specific time, the methods of the present invention significantly advance the characterization of chromosomes. The methods of the present invention have the ability to identify factors that would be difficult to uncover using standard genetic approaches.

Novel non-coding nucleic acids identified by the methods of the invention can be characterised for their normal biological activity via knock down strategies (described above) as well as via over expression in vivo. Hence, the invention also provides means for isolating novel non-coding nucleic acids that can then be used for diagnostic and therapeutic purposes. By way of example, non-coding RNAs shown to be involved in recruiting tumour suppressor complexes to the chromatin in normal cells but which are depleted in transformed cells can be reintroduced into cancer cells in order to induce apoptosis or inhibit cell proliferation.

The invention is further illustrated by the following non-limiting examples.

### EXAMPLES

The present inventors have devised a technique that isolates chromatin with associated bound factors in sufficient purity to allow identification of the proteins, polypeptides and non-coding nucleic acids at the targeted locus. By way of exemplification, proteins associated with medium repeat elements present in mammalian chromatin have been purified and identified directly using mass spectrometry. Non-coding nucleic acids can be identified via subcloning and standard sequencing methodologies. Applying this technology to human telomeres, 92% of all proteins previously shown to bind telomeres were identified, including proteins with low level expression. Many non-coding nucleic acids, including several in the region of around 100 nucleotides in length, were also identified.

The technique can be adapted to a variety of experimental situations, including viral integration sites, specific knockout cells, and post-drug treatment changes to name but a few. The method of the invention advantageously allows identification of changes in protein/non-coding nucleic acid complexes associated with a target chromatin site, without requiring prior knowledge of any of the proteins that may already be associated with that target site as is required in ChIP-based methodologies.

### Example 1

### 1. Preparation of a Chromatin Template

The chosen starting cells were HeLa S3 cells (a human cancer cell line). From suspension, cells in spinner flasks (20 litres culture at a density of 0.5-1 10⁶ cells/ml) were pelleted by centrifugation at 2500 g for 10 minutes at room temperature, and then immediately resuspended in cross-linking solution (200 ml for 10¹⁰ cells; crosslinking solution: 3% formaldehyde into 1×PBS (from formaldehyde 37%, methanol stabilized solution)). For adherent cells, the media was first discarded and crosslinking solution is immediately added to the plates (10 ml/15 cm plate). Cells were incubated in crosslinking solution for 30 minutes at room temperature.

Cells in suspension were spun down at 3200 g for 10 minutes at 4° C. and the supernatant discarded. The material was aliquoted into 4 Falcon tubes (usually 8 ml pellet/tube). For adherent cells the crosslinking solution was discarded and the plates washed twice with 1×PBS solution (standard phosphate buffered saline solution supplemented with 1 mM PMSF). A further 3 ml of cell scrapping solution (1×PBS; 0.05% Tween-20) was added per plate and the cells were pooled into Falcon tubes on ice.

The cells were then washed four times in PBS by resuspending the cell pellet in 1×PBS solution bringing the volume to 50 ml/tube, then spinning down at 3200 g for 10 minutes at 4° C. The supernatant was discarded each time and the final washed pellet was resuspended in sucrose solution (bringing the volume to 50 ml/tube). The solution was spun down at 3200 g for 10 minutes at 4° C., the supernatant discarded and the pellet brought up to a volume of 40 ml with sucrose solution.

The mixture was transferred to a 40 ml Dounce homogenizer on ice and dounced 20 times with a tightly fitting pestle. The dounced mixture was transferred to a fresh Falcon tube and spun down at 3200 g for 10 minutes at 4° C. The supernatant was discarded and the pellet resuspended in 50 ml of glycerol buffer. This step was repeated once and the pellet resuspended in an equal volume of glycerol buffer to that of the pellet (that is, if the pellet volume is 7.5 ml, 7.5 ml of glycerol buffer is added). The preparation can be aliquoted into 1.5 ml volumes (that is ˜0.5 109 cell equivalent per 1.5 ml Eppendorf tube) and is then either snap frozen liquid nitrogen and stored at −80° C. or taken to the next step for the target pull-down. For the purposes of the telomere sequence pull down 3.10⁹ cell equivalent is used, equivalent to six 1.5 ml Eppendorf tubes of the preparation.

The above chromatin preparation protocol is suitable for use with eukaryotic cells, particularly mammalian cells. However, it will be appreciated that the methods of the present invention are not restricted to identification of non-coding nucleic acid factors that are solely associated with eukaryotic genomic DNA and chromatin. Protocols for purification of genomic nucleic acid plus associated non-coding nucleic acid factors are also known in prokaryotes as well as in viruses.

### 2. Optimising the Chromatin Template for the Hybridisation Step

The next step is described in reference to a single 1.5 ml aliquot of the chromatin template preparation as the starting point.

Chromatin template from step 1 was pooled with the other aliquots in a 50 ml Falcon tube, bringing the volume to 50 ml with 1×PBS. The pooled mixture (now corresponding to the starting 6 aliquots) is spun down at 3200 g for 10 minutes at 4° C. and the supernatant is discarded. Then the pellet was resuspended in 50 ml of fresh LB3JD buffer (10 mM HEPES-NaOH pH 7.9; 100 mM NaCl; 2 mM EDTA pH 8; 1 mM EGTA pH 8; 0.2% SDS; 0.1% Sarkosyl; make fresh, keep at room temperature and add PMSF to 1 mM final concentration) at room temperature. The solution was spun down at 3200 g for 10 minutes at 4° C., to produce a resulting pellet with typical volume of ˜4.5 ml. To the pellet was added 4.5 ml of LB3JD buffer to resuspend. The resultant solution was divided into three ˜3 ml aliquots in Falcon 15 ml tubes. The solutions were sonicated on ice in a 4° C. cold room (Misonix 3000 sonicator with a micro-tip) The sonication parameters were: power setting 7, 15 seconds constant pulse, 45 seconds pause with a 7 minutes total process time. These conditions will usually give chromatin fragments that are about 2-4 kb in length which is a suitable size for the subsequent hybridisation and pull down steps. The sonicated solution was divided into 1 ml aliquots and spun down at 16000 g for 15 minutes at room temperature. The supernatants were pooled into a 15 ml Falcon tube. 400 μl aliquots of the sonicated chromatin solution were run each on S-400-HR gel filtration spin columns (Microspin™, GE Healthcare) at 800 g for 2 minutes so as to reduce the salt concentration in the solution.

The eluates from the columns were pooled in a 15 ml Falcon tube and incubated at 58° C. for 5 minutes, which helps to unmask any endogenously biotinylated proteins present in the soluble chromatin preparation. The pooled solution was cooled to room temperature. 0.5 ml of Ultralink Streptavidin bead slurry was equilibrated in, and washed twice with 10 ml of LB3JDLS buffer (10 mM HEPES-NaOH pH 7.9; 30 mM NaCl; 2 mM EDTA pH 8; 1 mM EGTA pH 8; 0.2% SDS; 0.1% Sarkosyl; make fresh, keep at room temperature and add PMSF to 1 mM final concentration) and spun down at 3200 g for 2 minutes. The supernatant was discarded leaving a slurry pellet of around 0.5 ml volume. The washed streptavidin beads were added to the pooled soluble chromatin solution and the mixture was incubated for 2 hours at room temperature on a nutator. The solution was spun down at 3200 g for 10 minutes at room temperature to remove most of the endogenously biotinylated proteins, and thereby reducing background in the later steps. The supernatant was saved and contains the ‘cleared’ soluble chromatin solution.

The chromatin solution was now ready for hybridization. It had the following spectrophotometric characteristics:
DNA Concentration (O.D.260): 1.5-2 mg/ml
O.D.260/O.D.280>=1.55.

These values were obtained when LB3JDLS buffer was used as the ‘blank’ solution.

The solubilised and cleared chromatin solution can be stored at 4° C.

### 3. Hybridization and Target Sequence Capture (Pull Down)

The solubilised chromatin samples obtained in the previous step were spun down for 15 minutes at 16 000 g at room temperature. The aliquots were pooled together to give about 5 ml of chromatin sample, to which was added 20% SDS to 0.02% final concentration (1/1000 of volume of chromatin sample).

To the chromatin solution was added 30 □l of the desired oligonucleotide probe sequence (from 100 μM stock solution, see part 4 for details of the probe). A control reaction containing a probe having a scrambled or randomised sequence was also run in parallel.

The hybridisation reaction was divided into approximately 34×150 □l aliquots which were placed in a thermocycler for the hybridisation step (HYBAID Px2, Thermo Fisher).

The hybridisation program was as follows:

25° C. for 3 minutes

70° C. for 6 minutes

38° C. for 60 minutes

60° C. for 2 minutes

38° C. for 60 minutes

60° C. for 2 minutes

38° C. for 120 minutes

25° C. final temperature

The hybridised samples were pooled back into 1.5 ml Eppendorf tubes and spun down at 16000 g for 15 minutes at room temperature so as to remove any precipitate that may have formed during the hybridization step.

1.2 ml of MyONE C1 magnetic streptavidin beads (Invitrogen) were placed in a 15 ml Falcon tube, to which 8.8 ml of LB3JD buffer was added to equilibrate them. The beads were immobilized on the magnetic stand and the supernatant was discarded. The beads were washed in 10 ml of LB3JD buffer, immobilized again and resuspended into 1.2 ml total volume of LB3JD buffer.

The supernatant from the spun down hybridised chromatin samples was transferred to two 15 ml Falcon tubes. To each tube was added 3.5 ml of milliQ water and then 0.6 ml of C1 magnetic bead solution. The mixtures were nutated for 12 hours at room temperature.

The volume of the mixtures was made up to 10 ml in each tube with LB3JD buffer. The magnetic beads were then immobilized on the magnetic stand. The supernatant (approximately 10 ml) represents the unhybridised fraction and can be saved for separate analysis later. The pellet represents the pulled down hybridised chromatin target. The pellet was washed seven times with 10 ml of LB3JD buffer. After each wash the beads were gently resuspended by nutation. Following the wash steps the beads were resuspended in 2.4 ml of LB3JD buffer per tube and transferred to 2 Eppendorf (1.5 ml) low binding tubes/pull-down.

The magnetic beads were immobilized using an Eppendorf magnetic stand. The supernatant was discarded and the beads were resuspended in 1 ml of LB3JD buffer. The tubes were incubated for minutes at 42° C. in a thermomixer (shaking at 1000 rpm) and then the beads were again immobilised using the Eppendorf magnetic stand. This step was repeated before the beads were finally resuspended in 0.6 ml of LB3JD buffer. The beads from each hybridization (telomere and control, 2 tubes each) were pooled in one tube/reaction and immobilized on the Eppendorf magnetic stand.

The beads were resuspended in 0.25 ml of Crosslink Reversal/Elution Buffer (XLRE Buffer: 10 mM NaOAc pH 5.5; 30 mM NaCl; 0.5 mM EDTA pH 8; 0.1 mM EGTA pH 8; 10 mM Hydrazine (from 11 M stock, neutralized with AcOH); 1% SDS) and incubated for two hours at 65° C. in a thermomixer shaking at 1200 rpm. The tubes were subjected to a brief spin to collect the solution and again placed on the magnetic stand. The eluate was retained and transferred to a clean 1.5 ml Eppendorf tube. The solution was adjusted to mildly basic conditions with Tris.HCl (25 μL, 1 M stock, pH 8.0) and 5 μL Proteinase K added (from 20 mg/mL stock). The tube was incubated at 42° C. for 30 min, shaking at 1200 rpm. The reaction was stopped by acidifying with NaOAc (25 μL of 3M NaOAc, pH 5.5) supplemented with approximately 20 μg of glycogen (1 μL, Sigma, glycogen from mussel, cat.1767). To the mixture was added 920 μL of ethanol, which was then incubated for one hour on dry ice in order to precipitate nucleic acid. The tube was spun down at 12000 g for 20 minutes at 4° C. and the supernatant was discarded. The pellet was rinsed with 100 μL of 80% ethanol, agitated and re-spun at 16000 g (5 min., 4° C.) to precipitate further. The supernatant was removed and the pellet allowed to air dry for 5 to 10 minutes. The pellet comprises genomic DNA as well as associated non-coding nucleic acid sequences and probe sequences. As such at this stage the pellet comprises pre-purified RICh material and can be further processed depending on the nature of the particular non-coding nucleic acids required for analysis.

In order to remove probe and genomic DNA the following steps may be followed. The pellet was resuspended in 48 μL DNase Buffer (1× Promega) supplemented with 1 μL Rnase inhibitor (RnasIN, Invitrogen) and 2 μL DNaseQ1 (Promega) and incubated at 37° C. for 30 min with shaking (1200 rpm). The reaction was quenched with 500 μL Trizol (Invitrogen), shaken and frozen at −20° C. overnight. The sample was then thawed samples and 100 μL CHCl₃ was added. The tube was mixed and allowed to stand for 5 min at room temperature. The phases were separated by centrifugation at 12000 g for 15 minutes at 4° C. The aqueous phase was then transferred to a fresh tube where the CHCl₃ extraction step was repeated. The aqueous phase was removed and added to 350 μL of isopropanol, shaken and incubated at room temperature for one hour. The remaining non-coding nucleic acids were then pelleted by centrifugation at 12000 g, for 20 minutes at 4° C. The supernatant was discarded and the pellet rinsed twice with 100 μL 80% ethanol. The pellet was centrifuged at 16000 g for 5 minutes at 4° C. and the supernatant was discarded. The pellet was allowed to air dry for 5 to 10 minutes before resuspension in 20 μL ddH2O. This represents a purified RICh material and following the last step of the protocol substantially comprises the population of non-coding RNAs (ncRNAs) that are associated with the specific region of chromatin targeted by the probes of the invention.

Sucrose Buffer

0.3 M Sucrose; 10 mM HEPES-NaOH pH 7.9; 1% Triton X-100; 3 mM CaCl₂; 2 mM MgOAc (Magnesium Acetate)

Glycerol Buffer 25% Glycerol; 10 mM HEPES-NaOH pH 7.9; 0.1 mM EDTA; 0.1 mM EGTA; 5 mM MgOAc (Magnesium Acetate)

### 4. Oligonucleotide Probe Design

The probes are constituted of a mixture of LNA and DNA residues and were 25 nucleotides long. In addition, they were labelled with the biotin analogue, desthiobiotin at their 5′ terminus, with the label being linked to the probe sequence via an extra-long spacer group, typically of between 20 and 95 carbon atoms in length. Desthiobiotin was selected as the affinity label so that more gentle elution conditions could be used compared to biotin. The extra-long spacer is of considerable importance, as the chromatin immobilization strategy could be impaired by steric hindrance problems (see for instance: Sandaltzopoulos R, Blank T, Becker P B. EMBO J. 1994). Suitable extra-long spacer technologies are described in Morocho et al. (Nucleos. Nucleot. Nuc. Acids. (2003) 22 (5-8):1439-41; and Methods Mol. Biol. (2005); 288:225-40).

In the example described above the telomere targeting probe sequence was:

[SEQ ID NO: 1]
5′ TtAgGgTtAgGgTtAgGgTtAgGgt 3′

The randomised/scrambled control probe sequence was:

[SEQ ID NO: 2]
5′ GaTgTgGaTgTggAtGtGgAtgTgg 3′

Where CAPITALIZED letters represent LNA residues and lower case letters were DNA residues. The desthiobiotinylated LNA containing oligonucleotide probes were synthesised to order by Fidelity Systems (Gaithersburg, Md., USA). The molecular spacer utilised in the above probes was 108 carbon atoms in length.

### Example 2

The starting cells were the transformed human embryonic lung fibroblast cell line WI38 VA13 (Castellani et al. (1986) J. Cell. Biol. 103:1671-1677). Unlike the HeLa cell line used in Example 1, W138 VA13 is a non-telomerase, ALT cell type. The ncRNA sequences associated with ALT telomere sequences were isolated according to the method described in Example 1.

Comparison of the ncRNAs isolated in Examples 1 and 2 can be made in order to determine whether there are differences in ncRNA populations at the telomeres of telomerase and non-telomerase human cell lines.

### Example 3

### Preparation of the Chromatin Template by Ultra-Sonication

The present example provides an alternative method for preparation of the chromatin fragments to that described in Example 1.

The following volumes and numbers are given for one purification (that is ˜3.10⁹ cell equivalent): Cells were centrifuged at 2000 g for 2 minutes at room temperature and pellet was resuspended into the same pellet volume of 1×PBS-0.5% Triton X-100 and 90 μl of RNaseA (Qiagen 100 mg/ml) were added. Cells were incubated for 60 minutes at room temperature with shaking then at 4° C. for 12-16 h. Cells were washed 6 times in PBS. Cells were equilibrated in LBJD solution (10 mM HEPES-NaOH pH 7.9; 100 mM NaCl; 2 mM EDTA pH 8; 1 mM EGTA pH 8; 0.2% SDS; 0.1% Sarkosyl, protease inhibitors) and pellet was resuspended into 55% pellet volume of LBJD solution. Samples were sonicated (Micro-tip, Misonix 3000) using the following parameters: Power setting 7 (36-45 Watts), 15 seconds constant pulse and 45 seconds pause for a 7 minutes total process time. Sample was collected by centrifugation at 16000 g for 15 minutes at room temperature. Chromatin sample was then applied to Sephacryl S-400-HR spin columns and incubated at 58° C. for 5 minutes.

LBJDLS (10 mM HEPES-NaOH pH 7.9; 30 mM NaCl; 2 mM EDTA pH 8; 1 mM EGTA pH 8; 0.2% SDS; 0.1% Sarkosyl, protease inhibitors) pre-equilibrated streptavidin beads were added (Pierce Ultralink streptavidin, 0.5 ml) and the sample was incubated for 2 h at room temperature. Beads were discarded and supernatant was saved.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

1. A method for isolating one or more non-coding nucleic acids associated with a target DNA sequence that is comprised within chromatin, comprising the steps of:
(a) obtaining a sample that comprises a target DNA sequence as well as one or more non-coding nucleic acids that are associated with the target DNA sequence;
(b) contacting the sample with at least one oligonucleotide probe that comprises a sequence that is complimentary to and capable of hybridising with at least a portion of the target DNA sequence, wherein the oligonucleotide probe comprises at least one modified nucleotide analogue and wherein the oligonucleotide probe further comprises at least one affinity label;
(c) allowing the at least one oligonucleotide probe and the target DNA sequence to hybridise with each other so as to form a probe-target hybrid;
(d) isolating the probe-target hybrid from the sample by immobilizing the probe-target hybrid through a molecule that binds to the at least one affinity label; and
(e) eluting the one or more non-coding nucleic acids that are associated with the target DNA sequence.

2. The method of claim 1 wherein the one or more non-coding nucleic acids comprises non-coding RNA.

3. The method of claim 1 wherein the one or more non-coding nucleic acids comprises one or more micro-RNAs.

4. The method of claim 1, wherein the at least one oligonucleotide probe comprises at least one group that conforms to general formula I, set out below:
A—[C]ₙ—X I
wherein A includes one or more affinity labels tethered to a modified nucleotide analogue X by a spacer group C of n atoms in length; A comprises a hapten or an immuno-tag; and wherein the nucleotide analogue X is selected from a peptide nucleic acid (PNA); a 2′ modified ribonucleotide analogue, including 2′-O—R sugar modifications, wherein R is selected from the group consisting of: methyl; ethyl; C₁ to C₅ alkyl; and aryl; a 2′ substituted ribonucleotide analogue, including 2′-C and 2′-deoxy-2′-halogeno, suitably 2′-deoxy-2′-fluoro ribonucleotides; and a morpholino nucleotide.

5. The method of claim 1, wherein the at least one oligonucleotide probe conforms to general formula II, set out below:
B—[C]ₙ—Y II
wherein B is an affinity label that is tethered to oligonucleotide sequence Y via a spacer group C comprising a linear chain of n atoms; the oligonucleotide sequence Y comprising at least 10 nucleotides of which no less than 10% are modified nucleotide analogues; typically at least 25% of the nucleotides are nucleotide analogues; and optionally up to 100% of the nucleotides are nucleotide analogues.

6. The method of claim 5, wherein the spacer group is linked to the 5′ nucleotide of the oligonucleotide probe.

7. The method of claim 5, wherein the nucleotide analogues are selected from 2′ modified ribonucleotide analogues, including 2′-O—R sugar modifications, wherein R is selected from the group consisting of: methyl; ethyl; C₁ to C₅ alkyl; and aryl.

8. The method of claim 5, wherein the nucleotide analogues are selected from 2′ substituted ribonucleotide analogues including 2′-deoxy-2′-halogeno, suitably 2′-deoxy-2′-fluoro ribonucleotides.

9. The method of claim 1, wherein the at least one oligonucleotide probe conforms to general formula III, set out below:
B—[C]ₙ—P III
wherein B is an immuno-tag or a hapten that is tethered to an peptide nucleic acid (PNA) sequence P via a spacer group C comprising a linear chain of n atoms.

10. The probe of claim 9, wherein the spacer group is linked to the N terminal residue of the PNA, P.

11. The probe of claim 9, wherein the spacer group is linked to the C terminal residue of the PNA, P.

12. The method of claim 1, wherein the at least one oligonucleotide probe conforms to general formula IV, set out below:
B—[C]ₙ-M IV
wherein B is an immuno-tag or a hapten that is tethered to a morpholino oligonucleotide sequence M via a spacer group C comprising a linear chain of n atoms.

13. The probe of claim 12, wherein the spacer group is linked to the 5′ nucleotide of the morpholino oligonucleotide sequence M.

14. The method of claim 1, wherein the at least one oligonucleotide probe comprises a group that conforms to general formula V, set out below: 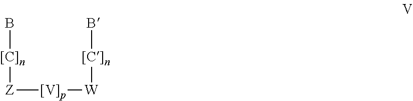
wherein B and B′ comprise an affinity label that is the same or different tethered to respective nucleotides Z and W by spacer groups C and C′ of n atoms in length; and
wherein the nucleotides Z and W are separated by an oligonucleotide chain T of p nucleotides in length, where p is between 0 and 40, the nucleotides Z, W and T being selected suitably from a ribonucleotide, a deoxyribonucleotide, a dideoxyribonucleotide and a modified nucleotide analogue,
the modified nucleotide analogue being selected from:
a locked nucleic acid nucleotide (LNA),
a 2′ modified ribonucleotide analogue, including 2′-O—R sugar modifications, wherein R is selected from the group consisting of: methyl; ethyl; C₁ to C₅ alkyl; and aryl; and
a 2′ substituted ribonucleotide analogue, including 2′-C, and 2′-deoxy-2′-halogeno, suitably 2′-deoxy-2′-fluoro ribonucleotides.

15. The method of claim 14 wherein the at least one oligonucleotide probe comprises the group of general formula III such that nucleotide Z represents the 5′ nucleotide in the oligonucleotide probe.

16. The method of claim 1, wherein the hapten is selected from the group consisting of: biotin or an analogue thereof, such as desthiobiotin; digoxigenin; fluorescein; and dinitrophenol.

17. The method of claim 1, wherein a plurality of oligonucleotide probes are used, and wherein each oligonucleotide probe hybridises to a different portion of the target DNA sequence.

18. The method of claim 1, wherein the target DNA sequence is comprised within one or more of the group consisting of: a telomere; a centromere; euchromatin; heterochromatin; intergenic regions; a gene; a repeat sequence; a heterologously inserted sequence; and an integrated viral genome.

19. A method of screening for a modulator of epigenetic activity comprising:
isolating a non-coding nucleic acid that is identified as associating with a specific region of chromatin in the genome of a eukaryotic cell according to the method of claim 1;
contacting the isolated non-coding nucleic acid with one or more compounds from a library of compounds; and
identifying those compound(s) that bind to and modulate the activity of the isolated non-coding nucleic acid as modulators of epigenetic activity.

20. A method of characterising the biological activity of a non-coding nucleic acid comprising the steps of:
isolating a non-coding nucleic acid that is identified as associating with a specific region of chromatin in the genome of a eukaryotic cell according to the method of claim 1;
generating an antisense nucleic acid sequence that is complementary to all or a part of the sequence for the a non-coding nucleic acid;
introducing the antisense nucleic acid sequence into a eukaryotic cell so as to deplete the endogenous level of the non-coding nucleic acid in the cell; and
analysing the phenotype of the eukaryotic cell so as to determine the biological activity of the a non-coding nucleic acid.

21. (canceled)

22. (canceled)

23. (canceled)

24. (canceled)

25. A nucleic acid analogue probe, suitable for use in PICh/RICh, as set out in formula I below:
A—[C]ₙ—X I
wherein A includes one or more affinity labels tethered to a nucleotide analogue X by a spacer group C of n atoms in length; A comprises a hapten or an immuno-tag; and wherein the nucleotide analogue X is selected from a peptide nucleic acid (PNA); a 2′ modified ribonucleotide analogue, including 2′-O—R sugar modifications, wherein R is selected from the group consisting of: methyl; ethyl; C₁ to C₅ alkyl; and aryl; and
a 2′ substituted ribonucleotide analogue, including 2′-C and 2′-deoxy-2′-halogeno, suitably 2′-deoxy-2′-fluoro ribonucleotides; and a morpholino nucleotide.

26. A nucleic acid analogue oligonucleotide probe, suitable for use in PICh/RICh, conforming to general formula II, set out below:
B—[C]ₙ—Y II
wherein B is an affinity label that is tethered to oligonucleotide sequence Y via a spacer group C comprising a linear chain of n atoms; the oligonucleotide sequence Y comprising at least 10 nucleotides of which no less than 10% are nucleotide analogues; typically at least 25% of the nucleotides are nucleotide analogues; and optionally up to 100% of the nucleotides are nucleotide analogues.

27. (canceled)

28. (canceled)

29. (canceled)

30. (canceled)

31. (canceled)

32. (canceled)

33. (canceled)

34. (canceled)

35. (canceled)

36. (canceled)


SEQUENCE LISTING (2 sequences)

SEQ ID NO: 1 - DNA, 25 bp, Artificial Sequence
  probe sequence
ttagggttagggttagggttagggt

SEQ ID NO: 2 - DNA, 25 bp, Artificial Sequence
  probe sequence
gatgtggatgtggatgtggatgtgg